SEQUENCE LISTING (3 sequences)

SEQ ID NO: 1 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 127 aa, synthetic construct
GPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 127 aa, synthetic construct
GPTGTGESKSPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

(19) United States
(12) Patent Application Publication
(10) Pub. No.: US 2026/0272870 A1
(43) Pub. Date: Sep. 17, 2026

(54) COMPOSITION FOR USE IN PREVENTION AND/OR TREATMENT OF HUMAN TRANSTHYRETIN AMYLOIDOSIS

(71) Applicants: Anders Olofsson, Umeå (SE); Intissar Anan, Umeå (SE); Sanduni Jayakodi Arachchilage Wasana Jayaweera, Umeå (SE)

(72) Inventors: Anders Olofsson, Umeå (SE); Intissar Anan, Umeå (SE); Sanduni Jayakodi Arachchilage Wasana Jayaweera, Umeå (SE)

(21) Appl. No.: 19/469,729

(22) PCT Filed: Mar. 26, 2024
(86) PCT No.: PCT/SE2024/050271

§ 371 Date: Sep. 26, 2025

(30) Foreign Application Priority Data
Mar. 27, 2023 (SE) 2350342-8

(51) Int. Cl. A61K 31/198; A61K 38/06

(52) U.S. Cl. CPC A61K 31/198; A61K 38/063

(57) ABSTRACT

Composition comprising a thiol and/or disulfide-modifying agent for use in dissolving and/or inhibiting formation of human transthyretin amyloid, wherein the thiol and/or disulfide-modifying agent is or comprises an amino acid or a peptide provided with at least one thiol group, at least one thioester group, or at least one disulfide group.

### TECHNICAL FIELD

The present invention relates to human transthyretin amyloidosis and a composition comprising a thiol and/or disulfide-modifying agent for use in dissolving and/or inhibiting formation of human transthyretin amyloid.

### BACKGROUND ART

Transthyretin (TTR) amyloidosis is a group of diseases caused by a pathological aggregation of the protein TTR, called amyloid. The clinical picture of TTR amyloidosis affects the heart and peripheral nerves in the form hypertrophic cardiomyopathy and polyneuropathy. The symptoms are arrhythmia, gastrointestinal problems, sensory and motor loss in the hands and feet. This disease is known colloquially as Skelleftesjukan, but in technical terms, it is called ATTRv amyloidosis regarding the hereditary form. The abbreviation stands for amyloid TTR where v stands for variant (i.e. a mutation in the protein). TTR is a 127-residue tetrameric extracellular plasma protein predominantly produced by the liver, but is also produced in the choroid plexus of the brain, in the retina of the eye, as well as by the glucagon and insulin producing alfa and beta cells in the islets of Langerhans. TTR functions as a transport protein for both the thyroxine hormone T4 and the retinol-binding protein (RBP).

The conversion of native TTR into an amyloidogenic fold is a process that requires a partial denaturation of the TTR and a dissociation of its tetrameric fold. The unfolded protein then proceeds into larger assemblies, forming a fibrillar structure. These plaques are composed of a tangle of regularly ordered fibrillar aggregates called amyloid fibers, a protein fold shared by other peptides, such as the prions associated with protein misfolding diseases and amyloid beta associated with Alzheimer's disease.

Amyloidogenic mutations in TTR lowers the thermodynamic and kinetic stability and, hence, enhance the conversion rate into the pathological aggregated form, amyloid. Today more than 130 mutations have been linked to TTR amyloidosis. Not everyone with a mutation, however, develops the disease but the risk increases.

In Sweden, about 8,000 individuals carry an amyloidogenic mutation in TTR, and around 450 develop the disease. Worldwide, the number is at least tenfold.

TTR amyloidosis can also develop without the protein mutation. This is called ATTR wt (wild type), and mainly affects older men by accumulation in the heart. ATTR wt amyloid is also involved in lumbar spinal stenosis, preeclampsia, and carpal tunnel syndrome, which are symptoms that can be seen in both ATTRv and ATTR wt.

TTR amyloidosis is a very serious disease and, left untreated, it leads to death within about ten years. Preventing the formation of TTR-amyloid is, hence, of great therapeutic interest. Today TTR amyloidosis is treated in three different ways: 1. Liver transplantation-when the mutated protein is mainly expressed in the liver. 2. Stabilizing ligands-TTR is a tetramer that binds thyroxine hormone. It is known that the tetramer must dissociate for amyloid to form. When TTR binds thyroxine hormone, the structure is stabilized in the native state, hence, preventing its conversion into the amyloid state. As thyroxine in the levels required would be toxic, analogues have been developed. Tafamidis also known as Vyndaqel® is a stabilizer approved for the treatment of TTR amyloidosis. 3. Gene therapy-RNA interference, which when incorporated into a cell has the ability to degrade the mRNA used for TTR expression, thereby reducing the production of the protein. Patisiran and Inotersen are two of examples used for such treatment.

These treatment strategies work and delay the progression of the disease by reducing the amount of TTR that is converted into the amyloid fold. Many patients, however, do not respond to these kind of treatments. These treatment strategies are also costly.

Although the histopathological features of TTR amyloidosis are striking, where deposits of TTR amyloid in tissue can be visualized using both immunological assays as well classical staining, it is today not fully elucidated which form of TTR that is causing the major tissue damage. Therefore, to avoid interfering with the homeostatic functions of amyloid proteins, a novel therapeutic approach would be desirable.

### SUMMARY OF THE INVENTION

It is an object of the present disclosure to provide a composition for use in dissolving and/or inhibiting formation of human transthyretin amyloid.

According to a first aspect, there is provided a composition comprising a thiol and/or disulfide-modifying agent for use in dissolving and/or inhibiting formation of human transthyretin amyloid, wherein the thiol and/or disulfide-modifying agent is or comprises an amino acid or a peptide provided with at least one thiol group, at least one thioester group, or at least one disulfide group.

Transthyretin (TTR) amyloidosis is a group of diseases caused by a pathological aggregation of the protein TTR, called amyloid, which affects the heart and peripheral nerves in the form of polyneuropathy. Symptoms are arrhythmia, gastrointestinal problems, sensory and motor loss in the hands and feet. This disease is known colloquially as Skelleftesjukan, but in technical terms it is called ATTRv amyloidosis. The abbreviation stands for amyloid TTR where v stands for variant (i.e. a mutation in the protein). TTR is a 127-residue extracellular plasma protein predominantly produced by the liver, but is also produced in the choroid plexus of the brain, in the retina of the eye, and by the glucagon and insulin-producing alfa and beta cells in the islets of Langerhans. TTR functions as a transport protein for the thyroxine hormone T4 and the retinol-binding protein (RBP).

The conversion of native TTR into an amyloidogenic fold is a process that requires a partial denaturation of the TTR and a dissociation of its tetrameric fold. The unfolded protein then proceeds into larger assemblies forming a fibrillar structure. These plaques are composed of a tangle of regularly ordered fibrillar aggregates called amyloid fibers.

Amyloidogenic mutations in TTR lowers the thermodynamic and kinetic stability and, hence, enhance the conversion rate into the pathological aggregated form, amyloid. Today more than 130 mutations have been linked to TTR amyloidosis. Not everyone with a mutation, however, develops the disease but the risk increases.

TTR amyloidosis can also develop without the protein mutation, this is then called ATTR wt (wild type), and mainly affects older men by accumulation in the heart. ATTR wt amyloid is also involved in lumbar spinal stenosis, preeclampsia and carpal tunnel syndrome, which are symptoms that can be seen in both ATTRv and ATTR wt.

TTR amyloidosis is a very serious disease and, left untreated, it leads to death within about ten years. Preventing the formation of TTR-amyloid is, hence, of great therapeutic interest. Today TTR amyloidosis is treated in three different ways: 1. Liver transplantation; 2. Stabilizing ligands, and 3. Gene therapy.

These treatment strategies work and delay the progression of the disease by reducing the amount of TTR that is converted into the amyloid fold. Many patients, however, do not respond to these kinds of treatments. These treatment strategies are also costly.

It is today not fully elucidated which form of TTR that is causing the major tissue damage. It has now been discovered that the formation of TTR-amyloid is enhanced by an initial formation of a non-native disulfide bond between subunits of TTR. To avoid interfering with the homeostatic functions of amyloid proteins, a composition comprising a therapeutically effective amount of a thiol and/or disulfide-modifying agent is here used for preventing and/or treating human TTR amyloidosis. The thiol and/or disulfide-modifying agent modulates the mechanism behind the formation of pathological aggregation of the protein TTR, which ultimately results in amyloid deposition in the body. Use of a thiol and/or disulfide-modifying agent could prophylactically prevent occurrence of pathological aggregation of the protein TTR, or could at least alleviate symptoms of the disease.

TTR contains a singly cysteine that is located in position 10 of its sequence after removal of the signal peptide (SEQ ID NO: 1:(127 AA): GPTGTGESKC PLMVKVLDAV RGSPAINVAV HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE). A disulfide bond between adjacent monomers in their native tetrameric structure is not possible as the distance between adjacent cysteines in the native tetramer exceeds more than 20 Å. The formation of a disulfide bond is, however, feasible within its amyloid form and based on a recent structure derived from ex vivo TTR fibrils (Schmidt M, et al. Nat Commun. 2019 Nov. 1; 10 (1): 5008. doi: 10.1038/s41467-019-13038-z) position 10 adopts a position that readily facilitates the formation of disulfide bonds.

In formation of the amyloids the formation of a disulfide bridge strongly drives the transition from a normal protein to these amyloid forms. In the path from a native structure towards its amyloid fold, TTR will reside as small oligomeric assemblies that in a continuous process grow into larger assemblies, ultimately forming amyloid deposits. Formation of disulfide bridges is a process that occurs in many proteins and chemically this process is controlled by what is called redox, a disulfide bridge is formed when the side chains of two nearby cysteines are bound together. This is an oxidation process, and it can be catalyzed in a few different ways. TTR naturally should not have a disulfide bridge, but in plasma from people with a mutation in TTR, misfolded TTR circulates, and here we have recently noted that these have a disulfide bridge.

We have seen that the mechanism by which TTR is converted into amyloidogenic oligomers can be greatly enhanced by an oxidative agent that enhances the formation of a non-native disulfide bond between two TTR-subunits. In stark contrast to previous reports, we have now discovered that promoting the formation of disulfide bonds also promotes the conversion into misfolded oligomeric TTR. The result can be seen as the formation of oligomers having an apparent size between 2-50 subunits.

By using a disulfide-modifying agent with ability to break the disulfide link between two TTR subunits a full conversion of misfolded transthyretin oligomers to the native state, is obtained. Hampering the formation of TTR-S—S-TTR formation by increasing the reducing environment, or by irreversibly block the thiol group will also reduce the formation of TTR amyloid.

Formation of misfolded TTR appears to take place in plasma. Inhibiting formation of such misfolded TTR may be easier than dissolving already formed misfolded TTR. If the thiol-modifying agent is present and active in plasma formation of disulfide between two subunits of TTR can be hampered. When inhibiting the formation of misfolded TTR, i.e. by increasing the reducing potential of the environment, an energy barrier does not have to be crossed, while dissolving already formed misfolded TTR includes such an energy barrier crossing. By this regimen, there is provided a novel, and highly selective and specific treatment to prevent in vivo formation of TTR-amyloid, including the formation of oligomer soluble assemblies.

The molecule TTR has a cysteine in position 10 in its sequence. The cysteine has a thiol group, —SH, which also can be written as TTR-SH. Two TTR-SH can react with each other and now TTR-S—S-TTR is formed. The TTR-S—S-TTR is suspected to be a pathological form and constituting a step towards formation of amyloid deposits, and elevated levels is found in the plasma from individuals having a mutation in TTR such as TTR-V30M. The TTR-S—S-TTR can however also be detected in individuals without mutations but generally the levels are lower.

By adding a stochiometric excess of a composition comprising a therapeutically effective amount of a substance that can prevent the formation of a disulphide bond between two subunits of TTR can according to the present finding also be used to effectively hamper the conversion into amyloid. A composition having the ability to block the free thiol, either reversible such as the oxidative reaction with a R—SH group resulting in the formation of a TTR-S—S—R adduct (where R can in principle be any molecule), or irreversible such as the reaction with reactive oxygen species such as the (OH·) radical or superoxide anion (O₂) generating the formation of a sulfone (TTR-SO₂), which hence hamper TTR from forming a disulfide bond between two subunits. Also the already formed TTR-S—S-TTR molecules can be further oxidized by reacting with oxygen hence creating a TTR-S—SO₃ known as TTR-S-sulfonation.

The addition of an amino acid or a peptide provided with at least one thiol group, R—SH, to the solution will create a more reducing environment and reduce the oxidation into disulfides as well as break already formed TTR-S—S-TTR disulfides. The addition of an amino acid or a peptide provided with at least one thioester group, R—C(═O)—S—R′, such as e.g. acetyl-CoA, where R and R′ represent organic groups, will after hydrolysis create a thiol and thus create a molecule with the ability to interfere and break already formed TTR-S—S-TTR disulfides. Instead of using an R—SH molecule or R—C(═O)—S—R′ to force the TTR to fold back into its proper shape, adding groups that are already oxidized, R—S—S—R′-molecules, will exchange with the TTR-S—S-TTR as well as the TTR—SH group and hence both inhibit the formation-as well as break already formed-disulfide-linked TTR-dimers (where R and R′ can in principle be any molecule). Such molecules can then react with the oxidized TTR-S—S-TTR molecules and then instead form TTR-S—S—R. This has the same effect as when adding a R—SH molecule, namely that two TTR molecules no longer stick together.

A phosphine-containing molecule, which does not contain any sulphur, may also effectively reduce R—SS—R′ groups.

Using a composition comprising a thiol or disulfide-modifying agent, the need for a normal biological drug “key-in-the-lock” effect is not needed, it is enough to modify the redox environment. Use of compounds comprising a thiol and/or disulfide-modifying agent has shown that the misfolded form of TTR is reduced and restores the levels of circulating oligomers to the levels of healthy controls.

Use of such a composition comprising a thiol and/or disulfide-modifying agent can then be used for dissolving and/or inhibiting formation of human transthyretin amyloid. Such composition is cheaper than treatment strategies used today. The composition can also be used on patients that do not respond to known treatments. The composition is less costly than the treatment strategies used today. Further, use of such a composition avoid interference with the homeostatic functions of amyloid proteins.

As long as the amino acid or peptide comprises at least one thiol group, at least one thioester group, or at least one disulfide group it can be used to dissolve or inhibit the formation of human transthyretin amyloid. By using such a disulfide-modifying agent with the ability to break the disulfide link between two transthyretin (TTR) subunits a full conversion of misfolded transthyretin oligomers to the native state, can be obtained. Hampering the formation of TTR-S—S-TTR formation by increasing the reducing environment, or by irreversibly blocking the thiol group will also reduce the formation of TTR amyloid.

The composition comprising the thiol and/or disulfide-modifying agent preferably has an LD50>500 mg/kg body weight (bw), such as >750 mg/kg bw or >1000 mg/kg bw, of an adult non-fasting rat (oral intake). LD50 is understood as the calculated/statistically derived single dose of the thiol and/or disulfide-modifying agent of the composition, which can be expected to kill 50% of the test population.

The amino acid may be any amino acid or derivative or analogue thereof with thiol and/or disulfide-modifying ability having at least one thiol group, at least one thioester group or at least one disulfide group. The peptide may be composed of 2 to 50 such amino acids or derivatives or analogues of such amino acids joined through amide formation involving the carboxyl group of each amino acid and the amino group of the next.

The thiol and/or disulfide-modifying agent may comprise at least one thiol group, at least one thioester group and/or at least one disulfide group.

The thiol and/or disulfide-modifying agent with at least one thiol group may be described by the general formula: R—SH (R may be any molecule).

The thiol and/or disulfide-modifying agent with at least one disulfide group may be described by the general formula R—S—S—R′ (R and R′ may be any molecules. R and R′ may be the same molecule or be different molecules.)

In a non-claimed embodiment, the thiol and/or disulfide-modifying agent has at least one phosphine group and may be described by the formula PR₁R₂R₃ wherein R₁, R₂, R₃ may be any molecule. R₁, R₂, R₃ may be the same molecule or may be different molecules.

The thiol and/or disulfide modifying agent may be a disulfide reducing agent.

The thiol and/or disulfide reducing agent may be the thiol and/or disulfide-modifying agent with at least one thiol group: R—SH, or an agent with at least one phosphine group.

The thiol and/or disulfide-modifying agent comprising at least one thiol group may be a hydroxyl-containing thiol.

The thiol and/or disulfide-modifying agent may comprise a linear, branched, isoform, ester, carboxylic ester, alkyl, alkenyl, acyl, aryl, heterocyclyl, alkoxy, hydroxy, sulphonyl, sulphinyl, sulphonylamino, halo, amine, hydrate, solvate, pro-drug, tautomer, isomer or pharmaceutically acceptable salt of a hydroxyl-containing thiol or analogue, or derivative thereof.

Examples of such thiol and/or disulfide-modifying agents are: dithiotreitol (DTT) or dithioerythritol (DTE) (DTT and DTE being epimers of each other), beta-mercaptoethanol, cysteine or cysteine analogues or derivatives thereof.

The thiol and/or disulfide-modifying agent comprising at least one thiol group may be an amino acid or derivative or analogue thereof.

The thiol and/or disulfide-modifying agent may be a cysteine or analogue or derivative thereof.

The thiol and/or disulfide-modifying agent may comprise a linear, branched, isoform, ester, carboxylic ester, alkyl, alkenyl, acyl, aryl, heterocyclyl, alkoxy, hydroxy, sulphonyl, sulphinyl, sulphonylamino, halo, amine, hydrate, solvate, pro-drug, tautomer, isomer or pharmaceutically acceptable salt of a cysteine or analogue, or derivative thereof.

Examples of such thiol and/or disulfide-modifying agents are: N-acetylcysteine, N-acetyl-D-cysteine, 2-mercapto-N-(1-oxopropyl)glycine, N-acetylhomocysteine, N-acetyl-2-methyl-L-cysteine, acetylpenicillamine, N-(1-oxopentyl)-L-cysteine, N-(2-chloroacetyl)-L-cysteine, N-(2,2-dimethyl-1-oxopropyl)-L-cysteine, N-propionyl-L-cysteine, N-(1-oxobutyl)-L-cysteine, N-(1-oxo-2-propen-1-yl)-L-cysteine, N-(1-oxohexyl)-L-cysteine, N-isobutyryl-L-cysteine, N-(3-methyl-1-oxobutyl)-L-cysteine, glyclyl-L-cysteine, L-alanyl-L-cysteine, N-(2-hydroxyacetyl)-L-cysteine, L-seryl-L-cysteine, N-(3-hydroxy-1-oxobutyl)-L-cysteine, L-cysteine-N-(2-hydroxy-1-oxopropyl), N-[(2S)-2-hydroxy-1-oxopropyl]-L-cysteine, 2-mercaptopropionyl-L-cysteine, 3-mercaptopropionyl-L-cysteine, N-(3-mercapto-2-methyl-1-oxopropyl)-L-cysteine, L-cysteinyl-L-cysteine, N-(4-mercapto-1-oxobutyl)-L-cysteine, N-(2-mercapto-1-oxopropyl)-L-cysteine, N-(2S)-2-mercapto-1-oxopropyl]-L-cysteine, N-[(2R)-2-mercapto-1-oxopropyl]-L-cysteine, N-carboxycarbonyl)-L-cysteine, N-acetyl-L-cysteine methyl ester, N-acetylcysteine ethyl ester, N-acetyl-L-cysteine isopropyl ester, (2R)-2-(acetylamino)-3-mercapto-N-methylpropanamide, glutathione, homocysteine, cysteine-glycine etc.

The thiol and/or disulfide-modifying agent comprising at least one disulfide group may comprise a cystine or analogue or derivative thereof.

The cystine derivative may be a decarboxylated derivative such as cystamine dihydrochloride and D-panthethine. The cystine derivative may be a dimer/oxidized derivative thereof such as oxidized glutathione.

The thiol and/or disulfide-modifying agent may comprise a linear, branched, isoform, ester, carboxylic ester, alkyl, alkenyl, acyl, aryl, heterocyclyl, alkoxy, hydroxy, sulphonyl, sulphinyl, sulphonylamino, halo, amine, hydrate, solvate, pro-drug, tautomer, isomer or pharmaceutically acceptable salt of a cystine or analogue, or derivative thereof.

Examples of such thiol and/or disulfide modifying agents with such cystine compound are: disulfide linked dimers of glutathione, D-Pantethine, cystamine dihydrochloride etc.

The thiol and/or disulfide-modifying agent comprising at least one phosphine group may be a polysubstituted phosphine or analogue or derivative thereof.

The polysbstituted phosphine may be a trialkylated phosphine.

An example is a trialkylated phosphine containing carboxylic acids, such as tris(2-carboxyethyl)phosphine (TCEP).

The thiol and/or disulfide-modifying agent provided with at least one thiol group may be or comprise cysteine, N-acetyl cysteine, homocysteine, glutathione or any combination thereof.

The thiol and/or disulfide-modifying agent provided with at least one disulfide group may be or comprise cystine, oxidized glutathione or a combination thereof.

The composition for use as described above may further comprising pharmaceutically acceptable adjuvants, excipients, solvents, and/or buffers.

The composition for use as described above may comprise the thiol and/or disulfide-modifying agent in a therapeutically effective amount of at least 100 nM

The therapeutically effective amount is dependent on which thiol and/or disulfide-modifying agent is used. The effective amount may be 100 nM-100 mM, or 1-1000 μM.

The composition for use as described above may be for prevention and/or treatment of human transthyretin amyloidosis symptoms selected from polyneuropathy, cardiomyopathy, spinal stenosis, preeclampsia, carpal tunnel syndrome, spinal stenosis and preeclampsia.

According to one aspect there is provided a composition for use as discussed above, comprising administering the composition to a subject by intralymphatic injection, injection directly into a lymph node, subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

The composition may be administered in an amount of 1-100 mg thiol and/or disulfide modifying agent/kg body weight.

According to one aspect there is provided a method of dissolving and/or inhibiting formation of human transthyretin amyloid in a patient by administering a composition comprising a thiol and/or disulfide-modifying agent to the patient, which thiol and/or disulfide-modifying agent is or comprises an amino acid or a peptide provided with at least one thiol group, at least one thioester group, or at least one disulfide group.

The method may comprise administering the composition comprising the thiol and/or disulfide-modifying agent to a patient in a therapeutically effective amount by intralymphatic injection, injection directly into a lymph node, subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

The composition comprising the thiol and/or disulfide-modifying agent may be administered in an amount of 1-100 mg thiol and/or disulfide-modifying agent/kg body weight.

Such a dosage could be administered to a patient at intervals of 1 time a week to 4 times a day, such once a day. The turnover for TTR is approximately 48 hours in the human body. Hence, the dose interval is preferably adapted to this turnover time. To maintain an effective level of the thiol and/or disulfide-modifying agent the use of formulations with a delayed release of the active substance in the body may be applied. Modified-release dosage forms are a type of medication that delivers drugs into the body over a prolonged period of time. These types of dosage forms are designed to release the drug in a controlled manner, either through a specific delivery mechanism or a particular formulation. This controlled release allows for a more consistent and sustained therapeutic effect, and often results in fewer side effects and a lower frequency of dosing. Sustained-release dosage forms are a specific type of modified-release dosage form that delivers a drug at a constant rate over a prolonged period of time. This is typically achieved through the use of specialized coatings or formulations that allow the drug to dissolve slowly in the gastrointestinal tract. Sustained-release dosage forms are often used to treat chronic conditions that require continuous drug delivery. While sustained-release dosage forms are a specific type of modified-release dosage form, there are many other types of modified-release dosage forms available as well. These include extended-release, delayed-release, and controlled-release dosage forms, each of which has its own unique delivery mechanism and formulation. Extended-release dosage forms, for example, are designed to release the drug over an extended period of time, typically several hours to a day. This can be achieved through a variety of mechanisms, such as specialized coatings or matrix formulations that control the rate of drug release.

### BRIEF DESCRIPTION OF THE DRAWINGS

FIGS. 1a and 1b show an in vitro analysis of recombinant transthyretin (TTR) V30M and TTR C10S expressed and purified from E. coli. The TTR V30M has a mutation at position 30 where the normal residue valine (V) has been exchanged for a methionine (M). The TTR V30M has a higher propensity to convert from its native state into the amyloid state. The TTR C10S has a mutation in position 10 where its normal cysteine (C) has been exchanged for a serine(S) thus blocking its ability to form disulfide bond.

Each protein was divided in two aliquots. The first was treated with diamide that catalyses the formation of disulfide bond, the second was treated with the reducing agent beta-mercaptoethanol. The proteins were then incubated at 37° C. in either Oh, FIG. 1a, or 24 h, FIG. 1b, followed by analysis using a sandwich ELISA assay.

FIG. 2 shows with Western blot analysis that TTR-oligomers contain a high fraction of non-native disulfide bonds between subunits of the TTR. Immunoprecipitated TTR-oligomers from ATTR V30M plasma were boiled in SDS-loading buffer with (+) and without (−) a reducing agent, beta-mercapoethanol, and separated on the SDS-PAGE.

FIG. 3a shows plasma from an ATTR wt (wild type) patient with verified amyloidosis, as a function of incubation with 10 mM of NAC at 37° C. for 24 h, followed by analysis of the presence of TTR oligomers using a sandwich ELISA assay. FIG. 3b, left panel, shows the presence of pathological oligomers in human plasma isolated from ATTRv carriers (no. 770-804) constituted by TTR V30M and a single TTR A45T (no. 802), and healthy controls of TTR wild type (no. 1-5). The right panel shows the corresponding samples after the addition of 10 mM NAC. All samples were incubated at 37° C. for 24 h followed by analysis for the presence of TTR oligomers using a sandwich ELISA assay.

FIG. 4 shows in vitro treatment of human plasma from a patient with a TTR-V30M mutation in the TTR protein with different concentrations of NAC, 0-500 μM. The ability of NAC at different concentrations to dissolve TTR-oligomers in plasma during 24 h, at 37° C. was evaluated using a sandwich ELISA assay.

FIG. 5 shows in vitro treatment of human plasma from a patient with a TTR-V30M mutation in the TTR protein with 1 mM homocysteine. The ability of homocysteine to dissolve TTR-oligomers during 24 h, at 37° C. was evaluated using a sandwich ELISA assay.

FIG. 6 shows in vitro treatment of human plasma from a patient with a TTR-V30M mutation in the TTR protein with different concentrations of glutathione (GSH), 0-600 M. The ability of GSH at different concentrations to dissolve TTR-oligomers during 24 h, at 37° C. was evaluated using a sandwich ELISA assay.

FIG. 7 shows in vitro treatment of human plasma from a patient with a TTR-V30M mutation in the TTR protein with different concentrations of cysteine, 0-600 μM. The ability of the cysteine at different concentrations to dissolve TTR-oligomers during 24 h, at 37° C. was evaluated using a sandwich ELISA assay.

FIG. 8 shows in vitro treatment of human plasma from a patient with a TTR-V30M mutation in the TTR protein with different concentrations of dithiothreitol (DTT), 0-600 μM. The ability of DTT at different concentrations to dissolve TTR-oligomers during 24 h, at 37° C. was evaluated using a sandwich ELISA assay.

FIG. 9 shows in vitro treatment of human plasma from a patient with a TTR-V30M mutation in the TTR protein with different concentrations of beta mecrcaptoethanol (BME), 0-600 μM. The ability of BME at different concentrations to dissolve TTR-oligomers during 24 h, at 37° C. was evaluated using a sandwich ELISA assay.

FIG. 10 shows in vitro treatment of human plasma from a patient with a TTR-V30M mutation in the TTR protein with different concentrations of cystine, 0-600 μM. The ability of cystine at different concentrations to dissolve TTR-oligomers during 24 h, at 37° C. was evaluated using a sandwich ELISA assay.

FIG. 11 shows in vitro treatment of human plasma from a patient with a TTR-V30M mutation in the TTR protein with different concentrations of oxidized glutathione (GSSG), 0-600 μM. The ability of GSSG at different concentrations to dissolve TTR-oligomers during 24 h, at 37° C. was evaluated using a sandwich ELISA assay.

FIG. 12 shows in vitro treatment of human plasma from a patient with a TTR-V30M mutation in the TTR protein with different concentrations of D-pantethine 0-600 μM. The ability of D-pantethine at different concentrations to dissolve TTR-oligomers during 24 h, at 37° C. was evaluated using a sandwich ELISA assay.

FIG. 13 shows in vitro treatment of human plasma from a patient with a TTR-V30M mutation in the TTR protein with different concentrations of cystamine dihydrochloride 0-600 M. The ability of cystamine dihydrochloride at different concentrations to dissolve TTR-oligomers during 24 h, at 37° C. was evaluated using a sandwich ELISA assay.

FIG. 14 shows in vitro treatment of human plasma from a patient with a TTR-V30M mutation in the TTR protein with different concentrations of tris(2-carboxyethyl)phosphine (TCEP), 0-600 μM. The ability of TCEP at different concentrations to dissolve TTR-oligomers during 24 h, at 37° C. was evaluated using a sandwich ELISA assay.

FIG. 15 shows in vitro treatment of human plasma from a patient with a TTR-V30M mutation in the TTR protein with different concentrations of alanine, 0-600 M. The ability of alanine at different concentrations to dissolve TTR-oligomers during 24 h, at 37° C. was evaluated using a sandwich ELISA assay.

FIG. 16 shows in vitro treatment of human plasma from a patient with a TTR-V30M mutation in the TTR protein with different concentrations of methionine, 0-600 M. The ability of methionine at different concentrations to dissolve TTR-oligomers during 24 h, at 37° C. was evaluated using a sandwich ELISA assay.

FIG. 17 shows in vitro treatment of human plasma from a patient with a TTR-V30M mutation in the TTR protein with different concentrations of serine 0-600 μM. The ability of serine at different concentrations to dissolve TTR-oligomers during 24 h, at 37° C. was evaluated using a sandwich ELISA assay.

FIG. 18 shows in vitro treatment of human plasma from a patient with a TTR-V30M mutation in the TTR protein with different concentrations of diflunisal 0-600 M. The ability of diflunisal at different concentrations to dissolve TTR-oligomers during 24 h, at 37° C. was evaluated using a sandwich ELISA assay.

FIG. 19 shows in vitro treatment of human plasma from a patient with a TTR-V30M mutation in the TTR protein with different concentrations of luteolin 0-600 μM. The ability of luteolin at different concentrations to dissolve TTR-oligomers during 24 h, at 37° C. was evaluated using a sandwich ELISA assay.

FIG. 20 shows oligomers in plasma in a TTR-V30M patient after oral intake of NAC corresponding to (70 mg/kg body weight). Plasma was collected at time 0, 2 h, 4 h, 8h, and 24 h and all samples were analyzed simultaneously using the described sandwich ELISA assay.

FIG. 21 shows oligomers in plasma in a TTR-V30M patient after oral intake of NAC corresponding to (70 mg/kg body weight). Plasma was collected at time 24 h and 168 hours and all samples were analyzed simultaneously using the described sandwich ELISA assay. (A control sample before NAC treatment is also shown.)

### DETAILED DESCRIPTION

A range of different compositions comprising different disulfide-modifying agents for use in dissolving and/or inhibiting formation of human transthyretin amyloid has been tested in vitro using plasma from healthy individuals and individuals with different TTR mutations. The tested disulfide-modifying agents comprised at least one thiol group, at least one disulfide group or at least one phosphine group. The results were compared with the two TTR tetramer stabilizing agents, diflunisal and luteolin, and with the amino acids alanine, serine and methionine.

A clinical study has also been performed by administering N-acetyl cysteine (NAC) to an individual with confirmed TTR mutation (V30M).

### EXPERIMENTAL

### Material and Methods

Recombinant TTR expressed in E. coli

Recombinant TTR-C10S and TTR-V30M, were obtained from AlexoTech AB (Umeå, Sweden). The TTR V30M has a mutation at position 30 where the normal residue valine (V) has been exchanged for a methionine (M). The TTR V30M has a higher propensity to convert from its native state into the amyloid state. The TTR C10S has a mutation in position 10 where its normal cysteine (C) has been exchanged for a serine(S) thus blocking its ability to form disulfide bond.

The sequence is defined by the native sequence of TTR without the signal peptide and corresponds to a total length of 127 amino acids. SEQ ID NO: 2: TTR V30M 127 AA: GPTGTGESKC PLMVKVLDAV RGSPAINVAM HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE

SEQ ID NO: 3: TTR C10S 127 AA:
GPTGTGESKS PLMVKVLDAV RGSPAINVAV HVFRKAADDT
WEPFASGKTS ESGELHGLTT EEEFVEGIYK VEIDTKSYWK
ALGISPFHEH AEVVFTANDS GPRRYTIAAL LSPYSYSTTA
VVTNPKE

Before use, the TTR mutants were dissolved in buffer A, containing PBS, 0.02% NaN₃ and 1 mM EDTA, and subjected to size-exclusion chromatography (Superdex 200 10/300 GL; GE Healthcare, Chicago, IL, USA) equilibrated in the same buffer and fractions of tetrameric TTR were collected. A correct molecular weight of TTR was verified by mass-spectroscopy regarding all variants (LC/MS TOF 6230B, Agilent, Santa Clara, CA, USA).

### Human Plasma

Plasma from ATTR-V30M gene carriers as well as TTR wt was collected using EDTA tubes. All plasma samples were buffered by the addition of a 10× phosphate buffer pH 7.4 to acquire a final phosphate concentration corresponding to 20 mM, and then frozen in liquid nitrogen following storage at −80° C. Before analysis using the plasma was thawed and serial diluted in 20 mM phosphate, 150 mM NaCl, 0.3% tween-20 (PBS-T) following analysis in the below-described ELISA. Samples were only thawed once.

### TTR-Oligomer Specific Sandwich ELISA Based on MAB39-44

The monoclonal antibody, MAB-39-44, detects in a highly specific manner structural changes deviating from its native fold, occurring in the edge of the TTR molecule represented by the C-strand. Reactivity of the antibody has been show to correlate with the exposure of epitopes normally hidden in the native fold but exposed within its amyloidogenic state. MAB 39-44 (AS16 3112) was obtained from Agrisera AB (Umeå, Sweden) and coated to clear microtiter plates, NUNC, Immunosorpt 96 well (Thermofisher, Uppsala, Sweden) in PBS for 1 hour. Thereafter, a blocking buffer was added comprising 2% dry milk dissolved in TRIS 20 mM, pH7.5, 150 mM NaCl, and 0.3% Tween. The plate was washed 3*5 min with TRIS 20 mM, pH7.5, 150 mM NaCl, and 0.3% Tween, hereinafter called the wash buffer. Plasma was diluted 0-1000 times in wash buffer and incubated for 1 hour. Thereafter, the plate was washed 3*5 min with wash buffer. A polyclonal rabbit anti-TTR antibody A0002 (DAKO, Odense Denmark). diluted 1:1000 in blocking buffer, was added and incubated for 1 hour. The plate was washed 3*5 min with wash buffer. An anti-rabbit antibody conjugated to HRP enzyme, (Agrisera, Umeå Sverige), diluted 1:10 000 in blocking buffer was added and incubated for 1 hour. The plate was washed 3*5 min in wash buffer. The reaction was developed with 3,3′,5,5′-Tetramethylbenzidine (TMB) HRP substrate (Agrisera, Umeå, Sverige). Absorbance was measured at 550 nm. Alternatively, an enhanced chemoluminscense substrate (Agrisera, Umeå, Sverige) was used and the luminescence measured.

### Western Blot Analysis

Proteins were heated for 10 minutes at 96° C. in SDS-loading buffer containing 20% glycerol, 0.1M Tris pH 6.8, and 4% sodium dodecylsulphate followed by separation on a 12% SDS-PAGE. After separation the proteins were transferred to a nitrocellulose membrane (Amersham, Solna, Sweden). Unspecific sites where blocked with 2% dry milk dissolved in 50 mM tris pH 7.4, 150 mM NaCl and 0.3% tween-20. Detection of TTR was performed using the rabbit anti-TTR antibody A0002 (DAKO, Odense, Denmark), and a goat anti-rabbit HRP conjugated antibody (Agrisera, Umeå, Sweden). Development was performed using enhanced chemoluminiscense (ECL) (Agrisera, Umeå, Sweden).

### In Vitro Treatment in Plasma

In vitro treatment of human plasma with and without mutation in the TTR protein were tested with different concentrations of disulfide-modifying agents:
a) thiol-containing: N-acetylcysteine (NAC), homocysteine, glutathione (GSH), cysteine, dithiothreitol (DTT), and beta-mercaptoethanol;
b) disulfide-containing: cystine, oxidized glutathione (GSSG), D-pantethine and cystamine dihydrocloride;
c) phospine-containing: tricarboxyl-phoshine (TCEP); and control agents:
d) amino acids without thiol group: alanine, methionine, and serine;
e) TTR-stabilizers: diflunisal and luteoline.
f) diamide (tetrametylazodikarboxamid) Concentrations of 0-1000 μM were tested and their ability to dissolve TTR-oligomers in plasma during 24 h, at 37° C. was evaluated using the above-described sandwich ELISA assay.

Briefly, plasma from an ATTR-V30M patient and an ATTRwt patient were incubated with the indicated concentration of substance for evaluation at 37° C. for 24 hours during gentle agitation. All substances were prior to the experiment dissolved in PBS at a 10× concentration and added to the plasma which caused a dilution corresponding to 10%. After 24 h the samples were analyzed using the sandwich ELISA based on MAB39-44 as described above.

### Human Clinical Trial

Two studies were designed as open label proof-of-concept studies based on the permit 96-315 $402/9612. The inclusion criteria involved; written consent to participate in the study; genetically proven TTR gene mutation. A single dose corresponding to 70 mg/kg of NAC was given orally. Plasma was isolated before initiation followed by a single dose corresponding to 70 mg/kg body weight.

In the first study, as a function of time, plasma was collected after 0, 2, 4, 8, 16, and 24 hours followed by analysis using the above-described ELISA. All samples were analysed on the same plate to facilitate comparison.

In the second study, plasma was isolated before initiation and then subsequently analysed after 48 hours and 168 hours. All samples were analysed on the same plate to facilitate comparison.

### Results

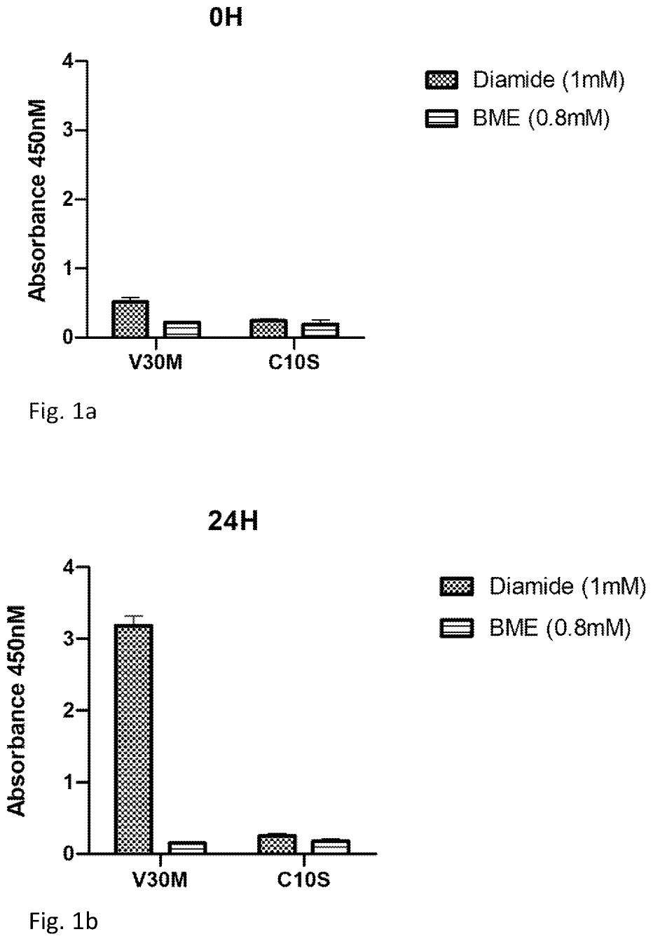

Using a sandwich ELISA based on the amyloid specific antibody MAB39-44 it is possible to probe the conversion of TTR from its native state into amyloid. We can here show that promoting the formation of a non-native disulfide link in TTR also strongly promote the exposure of epitopes normally buried within the native structure but exposed within the amyloid fold of TTR. This is shown in FIG. 1 where prolonged incubation of recombinant TTR-V30M in the presence of the disulfide catalyst diamide results in the exposure of a normally buried epitopes. We can here also show that in the presence of the reducing agent beta-mercaptoethanol this does not occur. The same effect is accomplished if the only cysteine is exchanged for serine. The presence of a reducing agent such as beta-mercaptoethanol consequently supress this effect while the effect is promoted by the disulfide bond catalyst diamide.

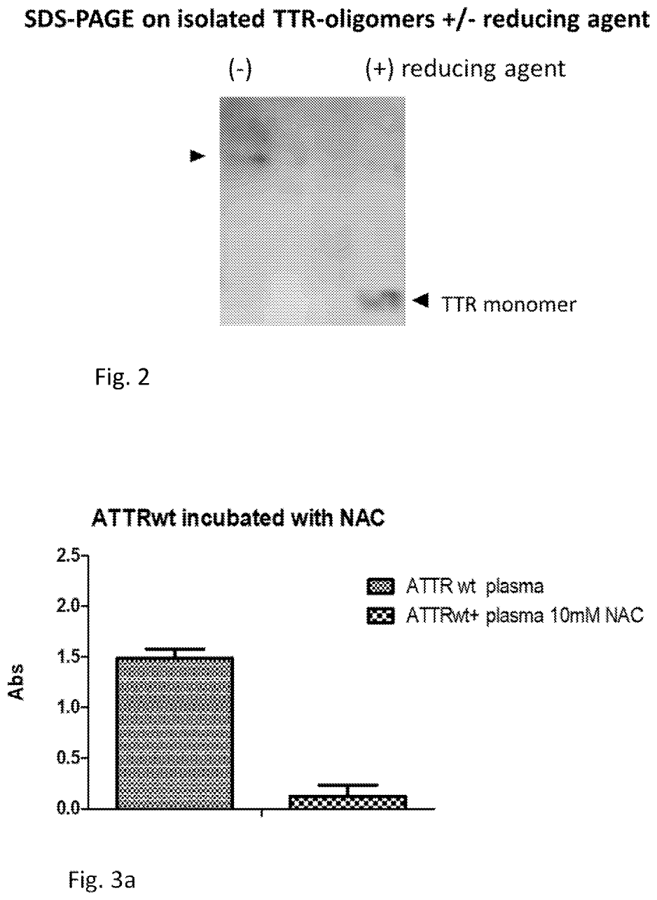

Using MAB39-44 non-native TTR-oligomers can also be detected in human plasma and through immunoprecipitation we can show that the non-native form of TTR contains a high proportion of a disulfide bond, shown in FIG. 2.

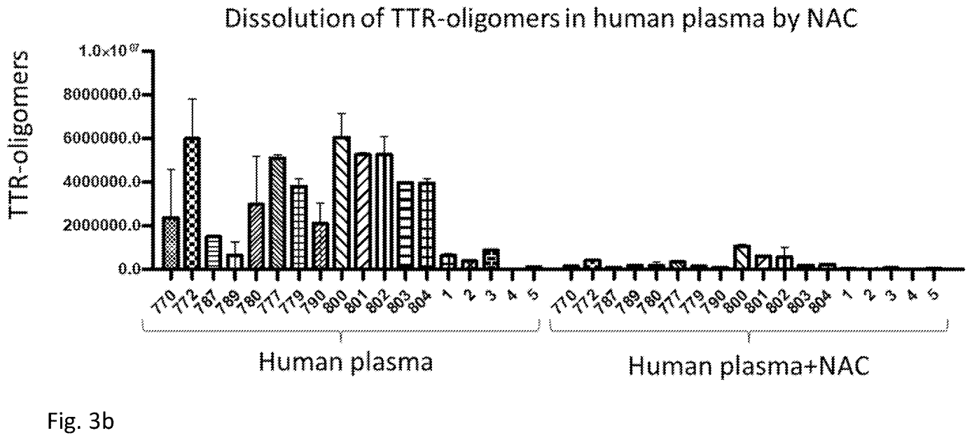

Using the sandwich ELISA based on the MAB39-44 the relative concentration of oligomers can be probed also in plasma. In FIG. 3A we here show that the concentration of oligomers is significantly enhanced in carriers of an ATTRv mutation and in plasma from a set of 12 individuals carrying the TTR V30M and one individual carrying the TTR A45T form, the levels of oligomers are elevated compared to healthy controls, represented by healthy TTR wt controls. We can also show that treatment with 10 mM of the reducing agent NAC, essentially fully eliminated the oligomeric forms of TTR. TTR-amyloidosis is also associated with the TTR wt form and FIG. 3B shows how the addition of NAC to plasma isolated from an ATTR wt patient also is eliminated.

N-acetylcysteine (NAC) is a well-known antioxidant, similar to A, C, E vitamins. In Suhr, O. B. et al. “Scavenger treatment of free radical injury in familial amyloidotic polyneuropathy: a study on Swedish transplanted and non-transplanted patients, Scand. J. Clin. Lab. Invest., 2001 February, Vol. 61, No. 1, pp 11-18, NAC is used to reduce 4-hydroxy-2-nonenal (HNE), a toxic product of lipid peroxidation, present in amyloid deposits in different forms of amyloidosis. The antioxidant effect of acetylcystein was used to prevent free oxygen and nitrogen radicals. The acetylcysteine was used against the secondary cellular effects that occur in transthyretin amyloidosis. The study did not reveal any dissolution of/inhibition of formation of human transthyretin amyloid.

The ability to restore the non-native form of TTR by reducing the non-native bond prompted us to probe if this is a specific effect of NAC or if this can be performed using any substance having the ability to reduce or alter this interaction in way that prevent subunits of TTR to form an intermolecular disulfide bond.

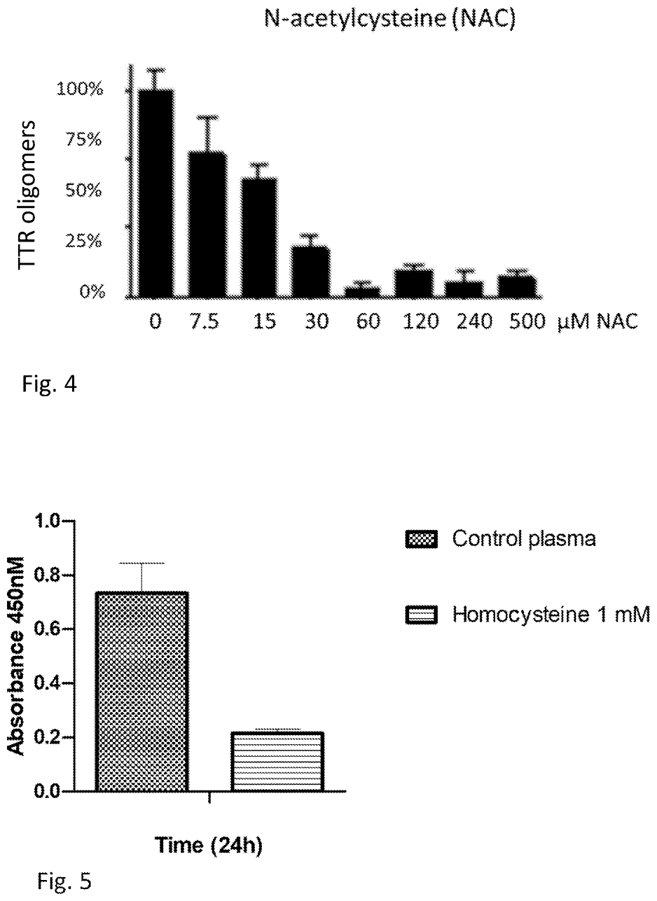

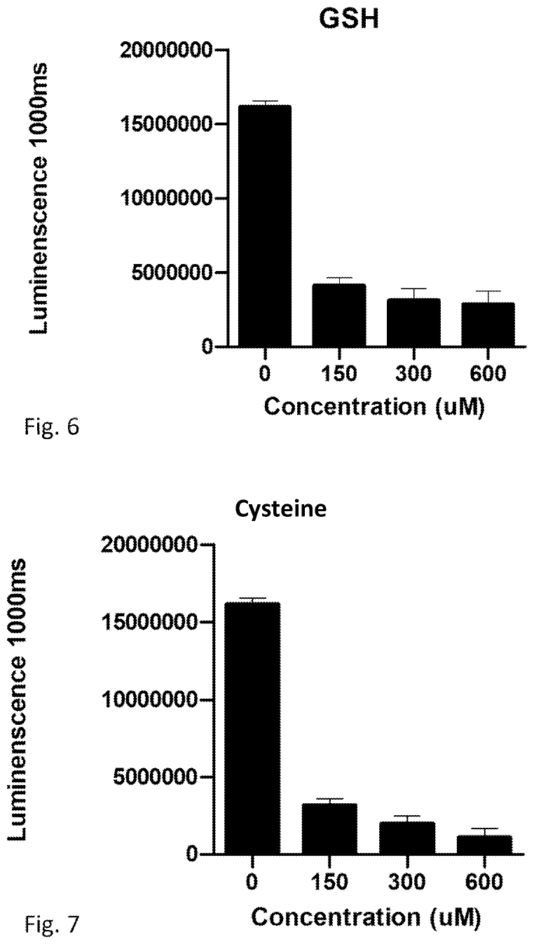

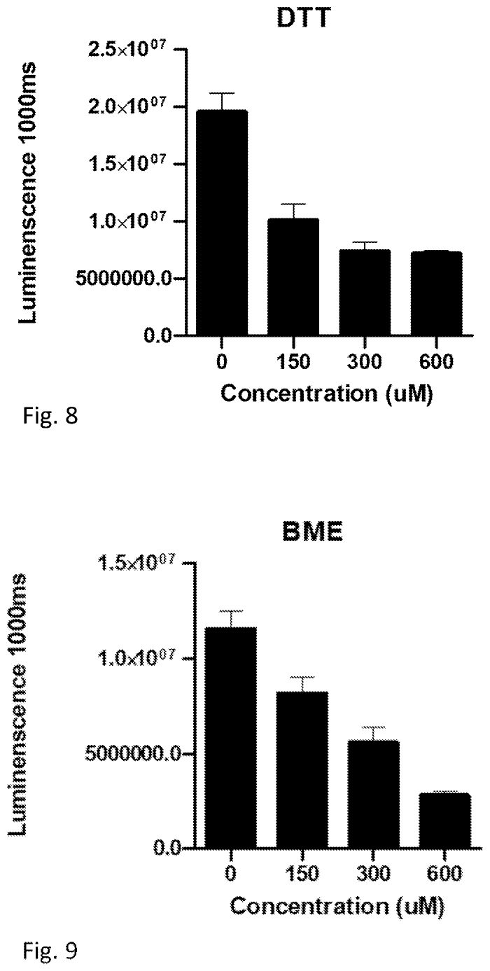

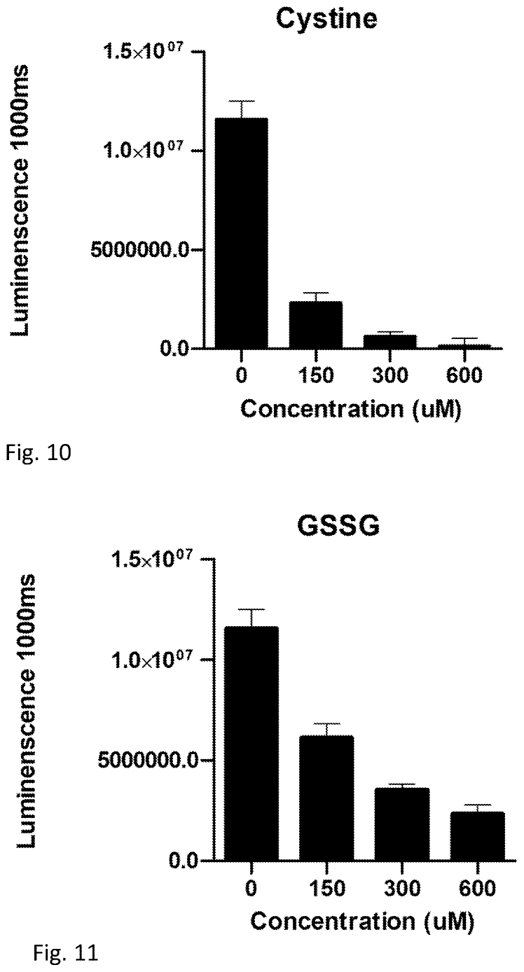

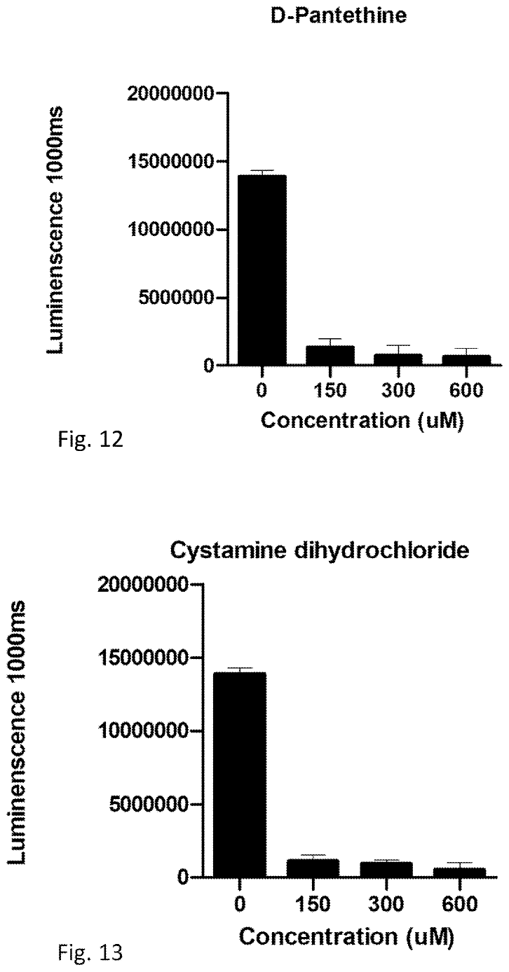

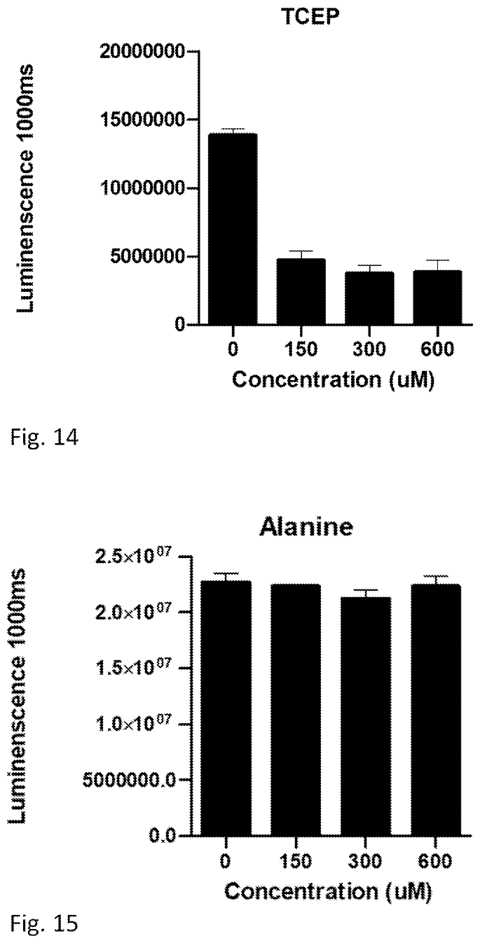

In vitro tests performed on human TTR V30M plasma showed that all compositions comprising a disulfide-modifying agent comprising at least one thiol group, at least one disulfide group or at least one phosphine led to decreased levels of non-native TTR-oligomers, FIG. 4-14.

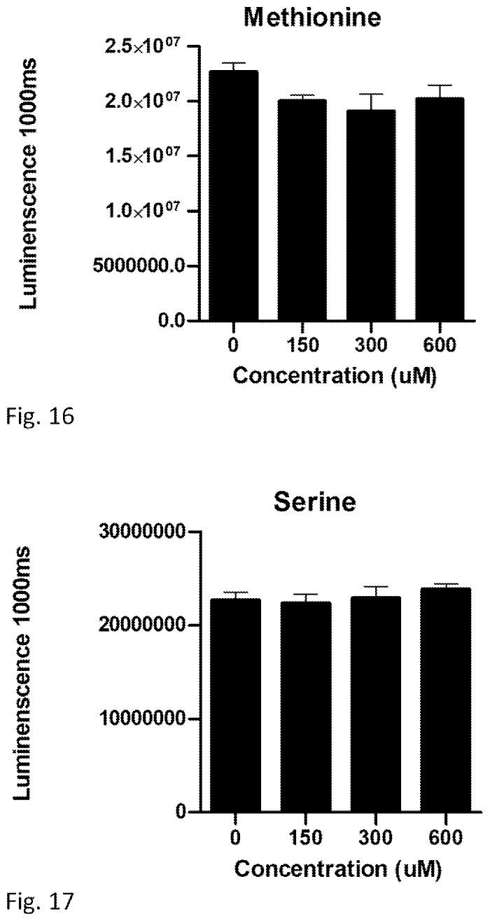

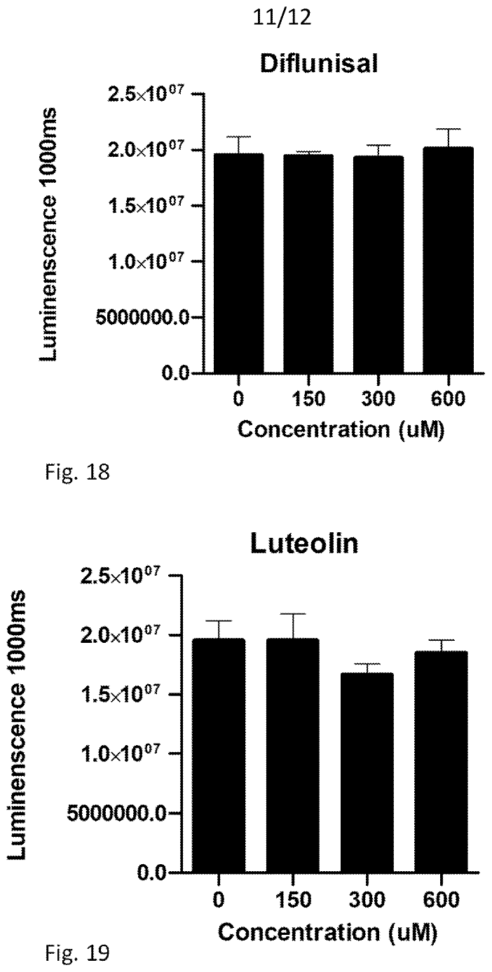

The results were also compared with the two TTR tetramer stabilizing agents, diflunisal and luteolin, and with the amino acids alanine, serine and methionine. These compounds did not result in any decrease of the non-native TTR-oligomers present in plasma, FIG. 15-19.

The results showed that disulfide-modifying compounds exert a strong suppression of the signal as a function of the compound. Different concentrations of the disulfide-modifying agents were also tested in vitro in plasma for most of the substances. A concentration dependent effect was noted regarding compositions comprising a disulfide-modifying agent comprising at least one thiol group, at least one disulfide group or at least one phosphine led to decreased levels of non-native TTR-oligomers. They all resulted in a significant suppression of the signal at concentrations below 600 UM and frequently also much lower. A detailed titration of NAC shows that it resulted in significant reduction of non-native TTR-oligomers already at 15 μM.

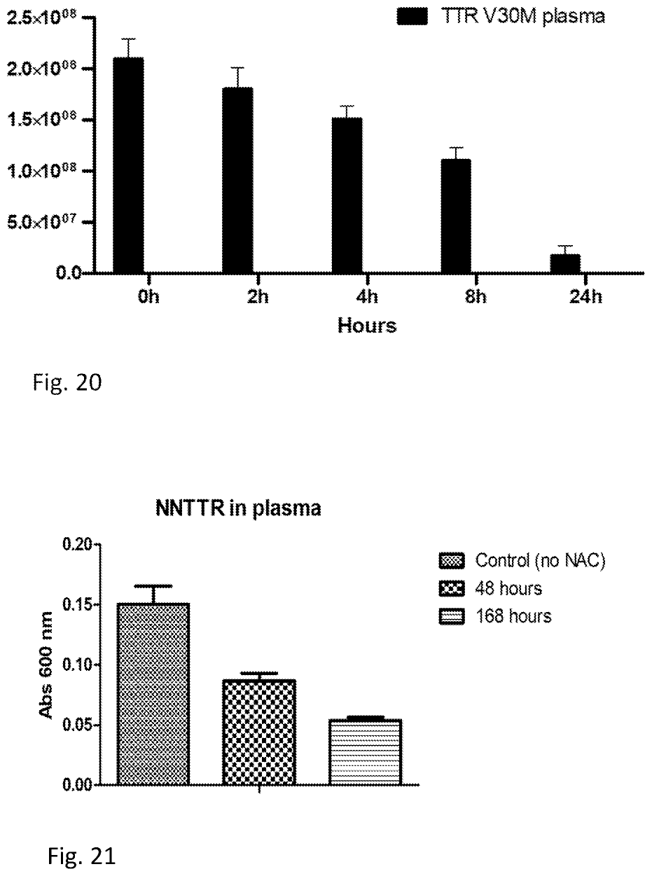

An effect of NAC was also noted in plasma after oral administration and FIG. 20 and FIG. 21 illustrate how the presence of oligomers after a single dose of NAC (70 mg/kg body weight) monitored by the amyloid-specific ELISA based on MAB39-44. The result indicates a decrease in the relative concentration of TTR-oligomers.

### DISCUSSION

The serendipic finding that a non-native disulfide bond in TTR locks the protein into an amyloidogenic oligomer presents a completely novel therapeutic avenue. We can here show that breaking this non-native disulfide bond relieves the constraints preventing a native fold and thus enable a conversion back to its native state. Any agent that can break this disulfide interaction consequently present a route to also restore the structure back to its native state. This is strongly supported by the finding that also vastly different reducing agents such as the various thiols, represented by NAC, GSH, DTT, BME, cysteine, and homocysteine restores the non-native oligomers back to its native state. In further support of this generic avenue also the non-thiol containing reducing agent phosphine, displays the same effect. Breaking the disulfide interaction between subunits of TTR can also be accomplished by the spontaneous disulfide exchange that frequently occurs between disulfides. Here we can show that the addition of an excess of small-molecules containing a disulfide bond also converts the non-native oligomers back to the native fold. This is here shown by GSSG, cystine, D-panthethine and cystamine dihydrochloride. In this context an adduct is instead added onto TTR but providing the added adduct is small and don't disturb the fold in a significant manner it will prevent misfolding and amyloid formation of TTR. It should be noted that the thiol group of TTR in vivo is to a major extent bound to, GSH, cysteine, cysteine-glycine as well S-sulfonated and only a small fraction of the thiol groups is unmodified.

Today more than 130 single point mutations in TTR are known to cause amyloidosis. We can here show that a thiol and/or disulfide-modifying agent efficiently restores that structure of oligomers derived from ATTR wt, ATTR V30M, and ATTR A45T. The path by which TTR converts into the amyloid fold is most likely generic and the approach of preventing the formation of a non-native disulfide bond between subunits of TTR can most likely be applied on all known amyloidogenic variants.

1. A composition comprising a thiol and/or disulfide-modifying agent for use in dissolving and/or inhibiting formation of human transthyretin amyloid, wherein the thiol and/or disulfide-modifying agent is or comprises an amino acid or a peptide provided with at least one thiol group, at least one thioester group, or at least one disulfide group.

2. The composition for use according to claim 1, wherein the thiol and/or disulfide-modifying agent provided with at least one thiol group is or comprises cysteine, N-acetyl cysteine, homocysteine, glutathione or any combination thereof.

3. The composition for use according to claim 1, wherein the thiol and/or disulfide-modifying agent provided with at least one disulfide group is or comprises cystine, oxidized glutathione or a combination thereof.

4. The composition for use according to claim 1, further comprising pharmaceutically acceptable adjuvants, excipients, solvents, and/or buffers.

5. The composition for use according to claim 1, comprising the thiol and/or disulfide-modifying agent in a therapeutically effective amount of at least 100 nM.

6. The composition for use according to claim 1, for prevention and/or treatment of human transthyretin amyloidosis symptoms selected from polyneuropathy, cardiomyopathy, spinal stenosis, preeclampsia, and carpal tunnel syndrome.

7. The composition for use according to claim 1, comprising administering the composition to a subject by intralymphatic injection, injection directly into a lymph node, subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

8. The composition for use according to claim 7, wherein the composition is administered in an amount of 1-100 mg thiol and/or disulfide modifying agent/kg body weight.

9. A method of dissolving and/or inhibiting formation of human transthyretin amyloid in a patient by administering a composition comprising a thiol and/or disulfide-modifying agent to said patient, which thiol and/or disulfide-modifying agent is or comprises an amino acid or a peptide provided with at least one thiol group, at least one thioester group, or at least one disulfide group.

10. The method according to claim 9, comprising administering the composition comprising the thiol and/or disulfide-modifying agent to the subject by intralymphatic injection, injection directly into a lymph node, subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

11. The method according to claim 9, wherein the composition comprising the thiol and/or disulfide-modifying agent is administered in an amount of 1-100 mg thiol and/or disulfide modifying agent/kg body weight.
